(19) United States
(12) Patent Application Publication
(10) Pub. No.: US 2007/0238889 A1
(43) Pub. Date: Oct. 11, 2007

(54) PROCESS FOR PREPARING DC-CHOLESTEROL

(76) Inventors: Raghavendracharyulu Venkata Palle, Hyderabad (IN); Sekhar Munaswamy Nariyam, Hyderabad (IN); Raghupati Rama Subrahmanyam Vinjamuri, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/696,808

(22) Filed: Apr. 5, 2007

Related U.S. Application Data
(60) Provisional application No. 60/820,191, filed on Jul. 24, 2006.

(30) Foreign Application Priority Data
Apr. 7, 2006 (IN) 645/CHE/2006

(51) Int. Cl. C07J 9/00

(52) U.S. Cl. 552/544

(57) ABSTRACT

Processes for preparing 3β-[N-(N′,N′-dimethylaminoethane)-carbamoyl]cholesterol (“DC-cholesterol”) and its salts.

The present invention relates to a process for the preparation of DC-cholesterol and salts thereof.

DC-cholesterol has a chemical name 3β-[N-(N′,N′-dimethylaminoethane)-carbamoyl]cholesterol (hereinafter referred to by the adopted name “DC-cholesterol”) and is represented by structural Formula I.

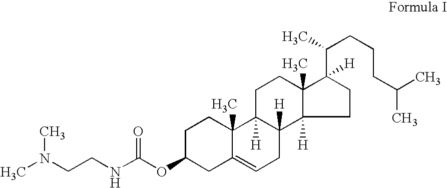

DC-cholesterol hydrochloride is one of the cationic lipids used in the preparation of liposomes in combination with DOPE (Dioleoyl phosphatidyl ethanolamine), for the delivery of nucleic acid fragments in gene therapy. This is a conceptually novel therapeutic strategy for the treatment and cure of acquired diseases like cancer and inherited diseases such as cystic fibrosis.

U.S. Pat. No. 5,753,262 discloses DC-cholesterol hydrochloride and a process for preparing the same. The process for the preparation of DC-cholesterol comprises the reaction of the cholesteryl chloroformate (46.45 mmol) with N,N-dimethylethylenediamine (278.4 mmol), in chloroform. The obtained material is crystallized in a mixture of ethanol and acetonitrile, then from cyclohexane and again the material was crystallized from ethanol and acetonitrile. This patent also discloses a process for preparation of DC-cholesterol hydrochloride comprises the treatment of DC-cholesterol with hydrogen chloride in ethyl acetate.

U.S. Pat. No. 6,319,516 discloses a process for the preparation of DC-cholesterol. The process comprises the reaction of cholesteryl chloroformate (0.5 mmol) with N,N-dimethylethylenediamine (9.1 mmol), in chloroform. The obtained DC-cholesterol was recrystallized from ethanol.

Chemical Abstracts, Vol. 132, Abstract No. 122799 (1999) summarizes an article by Yu et al. [in Chinese Pharmaceutical Journal (Taipei), Vol. 51(3), pp. 241-244, 1999] teaching a modified process, wherein cholesteryl chloroformate was reacted with N,N-dimethylethylenediamine in dry methylene chloride. A product yield of 73.2% was reported, as compared to a yield of 21.8% when dry chloroform was used.

The aforementioned processes suffer from serious disadvantages such as formation of impurities which may be due to the use of excess N,N-dimethylethylenediamine, rendering the processes unsuitable for industrial scale manufacturing.

A. Bianco et al., Letters in Organic Chemistry, Vol. 2, pages 79-82, 2005, describes a laboratory-scale synthesis of DC-cholesterol and its analogues that does not involve phosgene derivatives.

There remains a need for a safe and efficient industrial scale process for preparing DC-cholesterol and its salts free of the above-mentioned problems.

### SUMMARY OF THE INVENTION

The present invention relates to a process for the preparation of DC-cholesterol and salts thereof.

In one aspect of the present invention relates to a process for preparing DC-cholesterol, comprising reacting cholesterol, N,N-dimethylethylenediamine and 1,1′-carbonyldiimidazole.

In another aspect the present invention relates to a process for preparing DC-cholesterol, comprising reacting cholesterol, N,N-dimethylethylenediamine and triphosgene.

In further aspect of the present invention relates to a process for preparing DC-cholesterol, comprising reacting cholesteryl chloroformate with N,N-dimethylethylenediamine, wherein a molar ratio of cholesteryl chloroformate to N,N-dimethylethylenediamine is about 1:1 to about 1:5.

In yet another aspect, the present invention provides a process for the preparation of DC-cholesterol hydrochoride comprising reacting DC-cholesterol with hydrochoric acid in the presence of suitable solvent.

The process of the present invention is simple, improved, efficient, industrially feasible, and ecofriendly reproducing the desired compound of Formula I with high yield and purity.

### BRIEF DESCRIPTION OF THE DRAWINGS

FIG. 1 is an X-ray powder diffraction pattern of DC-cholesterol hydrochloride prepared according to Example 4.

FIG. 2 is a differential scanning calorimetry curve of DC-cholesterol hydrochloride prepared according to Example 4.

FIG. 3 is a thermogravimetric analysis curve of DC-cholesterol hydrochloride prepared according to Example 4.

FIG. 4 is an infrared absorption spectrum of DC-cholesterol hydrochloride prepared according to Example 4.

### DETAILED DESCRIPTION OF THE INVENTION

The present invention relates to a process for the preparation of DC-cholesterol and salts thereof.

In one aspect of the present invention relates to a process for preparing DC-cholesterol, comprising reacting cholesterol of Formula III, N,N-dimethylethylenediamine, and 1,1′-carbonyldiimidazole.

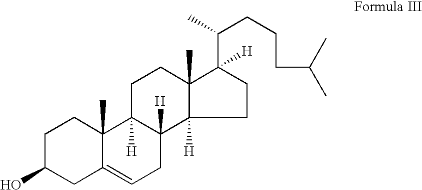

Molar ratios of cholesterol to N,N-dimethylethylenediamine are about 1:1 to about 1:5 in the preparation of DC cholesterol.

The quantity of 1,1′-carbonyldiimidazole, which is used in the preparation of DC cholesterol, is in the range of about 1 to about 5 moles per molar equivalent of cholesterol.

Suitable solvents, which can be used in the preparation of DC-cholesterol include but are not limited to dichloromethane, dichloroethane, chloroform and the like.

The temperature for preparing DC-cholesterol range from about −20 to 50° C., or about −5 to 10° C., depending on the solvent used.

After completion of the reaction the reaction mixture is washed with water and concentrated completely to afford solid. Concentration of the solvent can be conducted at temperatures from about 35° C. to about 60° C.

Another aspect of the present invention relates to a process for preparing 3β-[N-(N′,N′-dimethylaminoethane)-carbamoyl]cholesterol, comprising reacting cholesterol of Formula III, N,N-dimethylethylenediamine and triphosgene in the presence of a base.

Molar ratios of cholesterol to N,N-dimethylethylenediamine is about 1:1 to about 1:8 or about 1:7 in the preparation of DC cholesterol.

The quantity of triphosgene, which is used in the preparation of DC cholesterol, is in the range of about 1 to about 3 moles, per molar equivalent of cholesterol.

Suitable bases which can be used include but are not limited to sodium hydroxide, potassium hydroxide, sodium carbonate, sodium bicarbonate, potassium carbonate, potassium bicarbonate, triethylamine, diethylisopropylamine, diisopropyl ethylamine, methylamine, ammonia, and the like.

Suitable solvents, which can be used in the preparation of DC-cholesterol include but are not limited to dichloromethane, dichloroethane, chloroform and the like.

Suitable temperatures for preparing DC-cholesterol range from about −20 to 50° C., or about −5 to 10° C.

After completion of the reaction, the reaction mixture is washed with water and concentrated completely to afford a solid. Concentration of the solvent can be conducted at temperatures from about 35° C. to about 60° C.

In further aspect, the present invention relates to a process for preparing 3β-[N-(N′,N′-dimethylaminoethane)-carbamoyl]cholesterol, comprising reacting cholesteryl chloroformate of Formula II with N,N-dimethylethylenediamine, wherein a molar ratio of cholesteryl chloroformate to N,N-dimethylethylenediamine is about 1:1 to about 1:5.

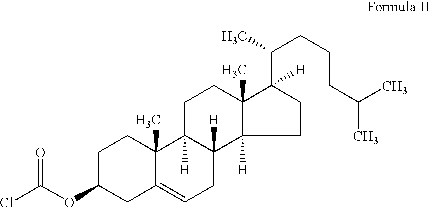

Suitable solvents, which can be used in the preparation of DC-cholesterol, include but are not limited to dichloromethane, dichloroethane, chloroform and the like.

The addition of cholesteryl chloroformate into N,N-dimethylethylenediamine solution, or the addition of N,N-dimethylethylenediamine solution into cholesteryl chloroformate, can be completed in times from about 15 minutes to about 2 hours, or longer.

Suitable temperatures for preparing DC-cholesterol range from about −20 to 50° C., or about −5 to 10° C.

After completion of the reaction, the reaction mixture is washed with water. The organic layer is combined with an anti-solvent and concentration is carried out to obtain suspension.

Suitable anti-solvents include n-hexane, n-heptane, cyclohexane, petroleum ether and the like.

Concentration for removing solvent can be carried out suitably using evaporation, atmospheric distillation or distillation under vacuum with stirring or without stirring of the solution. Concentration of the solvent can be conducted at temperatures from about 35° C. to about 60° C.

DC-cholesterol can be converted to desired pharmaceutically acceptable acid addition salts, by a process comprising reacting DC-cholesterol with a suitable acid, in the presence of a suitable solvent.

Suitable solvents, which can be used for dissolving DC-cholesterol either alone or combination with a pharmaceutically acceptable acids include but are not limited to: water; ethers such as diethyl ether, dimethyl ether, diisopropyl ether, methyl isobutyl ether, 1,4-dioxane, tetrahydrofuran, and the like; hydrocarbons such as such as toluene, xylene, n-pentane, n-hexane, n-heptane, cyclohexane, and the like; halogenated solvents such as dichloromethane, 1,2-dichloroethane, chloroform, carbon tetrachloride and the like; esters such as n-propyl acetate, n-butyl acetate, isobutyl acetate, t-butyl acetate and the like; alcohols such as methanol, ethanol, isopropyl alcohol, n-propanol, and the like; ketones such as acetone, ethyl methyl ketone, methyl isobutyl ketone and the like; nitriles such as acetonitrile and the like; and mixtures thereof.

Suitable pharmaceutically acceptable acids, which can be used, include, but are not limited to: inorganic acids such as hydrochloric acid, hydrobromic acid, hydroiodic acid; and organic acids such as acetic acid, tartaric acid, oxalic acid, methane sulphonic acid and the like.

In an embodiment, the present invention provides a process for the preparation of DC-cholesterol hydrochoride comprising reacting DC-cholesterol with hydrochoric acid in the presence of a suitable solvent.

Suitable solvents, which can be used for dissolving DC-cholesterol either alone or combination with a pharmaceutically acceptable acid include but are not limited to: water; ethers such as diethyl ether, dimethyl ether, diisopropyl ether, methyl isobutyl ether, 1,4-dioxane, tetrahydrofuran, and the like; hydrocarbons such as such as toluene, xylene, n-pentane, n-hexane, n-heptane, cyclohexane, and the like; halogenated solvents such as dichloromethane, 1,2-dichloroethane, chloroform, carbon tetrachloride and the like; esters such as n-propyl acetate, n-butyl acetate, isobutyl acetate, t-butyl acetate and the like; alcohols such as methanol, ethanol, isopropyl alcohol, n-propanol, and the like; ketones such as acetone, ethyl methyl ketone, methyl isobutyl ketone and the like; nitriles such as acetonitrile and the like; and mixtures thereof.

The dissolution temperatures can range from about 20 to 120° C., depending on the solvent used for dissolution. Any other temperature is also acceptable as long as a clear solution of DC-cholesterol is provided.

The quantity of solvent used for dissolution depends on the solvent and the dissolution temperature adopted. The concentration of DC-cholesterol in the solution may generally range from about 0.01 to about 0.2 g/ml, or about 0.1 g/ml in the solvent.

DC-cholesterol and HCl can be dissolved either in the same solvent or they may be dissolved in different solvents and then combined to form a mixture.

HCl can be used in the form of aqueous or alcoholic solutions such as aqueous HCl, methanolic HCl, isopropanolic HCl and the like, or HCl gas.

The obtained solution is immediately converted into a suspension and the solid is isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until the desired product purity is obtained; frequently times from about 1 to 20 hours, or longer, are used.

DC cholesterol obtained by the above process has been analyzed using high performance liquid chromatography (“HPLC”) with the conditions described in Table 1.

TABLE 1

| Column and | Zorbax CN 250 * 4.6 mm, 5μ | |
|---|---|---|
| Packing: | | |
| Buffer | About 9.625 g of ammonium acetate salt is | |
| | dissolved in 500 ml of water to give 0.25 M | |
| | ammonium acetate solution | |
| Sample | 20 mg of the sample is dissolved in 100 ml of mobile | |
| preparation | phase. | |
| Flow rate: | 1.0 ml/minute | |
| Detector | ELSD (Evaporated light scattering Detector) | |
| | Drift Tube | 80° C. |
| | Temperature | |
| | Gas Flow | 3.0 L/minutes |
| | Impactor | Off mode |
| | Gains | 1 |

| | |
|---|---|
| Temperature: | 30° C. |
| Injection volume: | 50 μL |
| Mobile phase | Methanol and buffer mixed in the ratio of 90:10. |
| | About 5 ml of triethylamine was added to 1 liter of |
| | mobile phase, mixed well, filtered, and degassed. |

The DC-cholesterol hydrochloride obtained by the process of present invention typically has a purity of not less than about 90%, or about 95%, as determined by high performance liquid chromatography (HPLC).

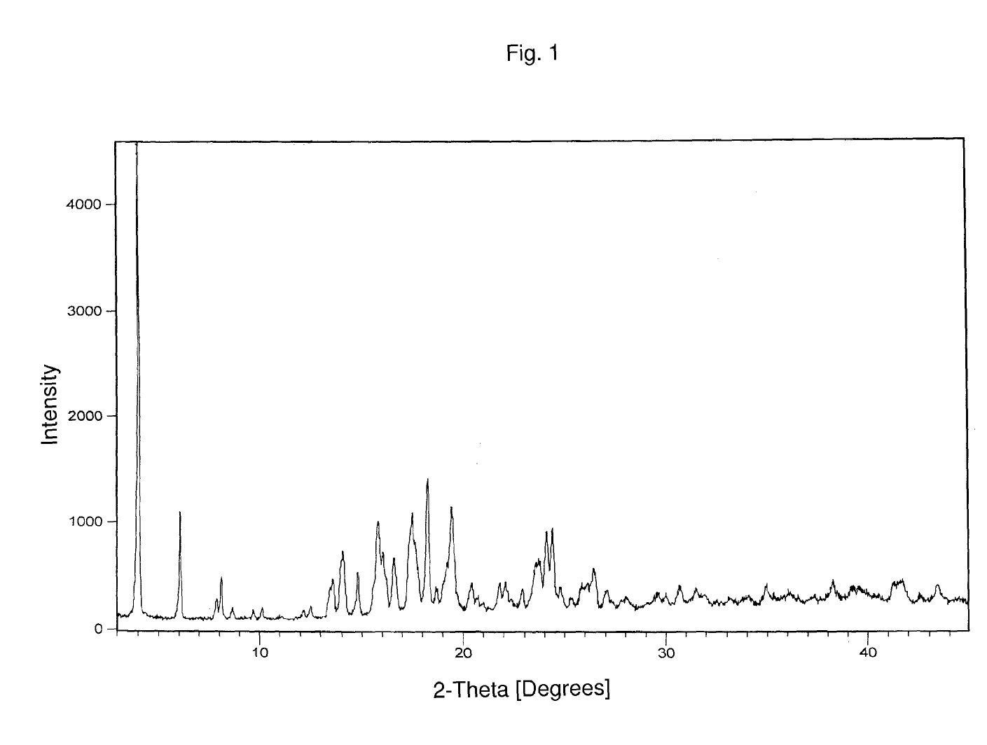

Crystalline DC-cholesterol hydrochloride obtained in the process of present invention is characterized by its XRPD pattern, substantially in accordance with the pattern of FIG. 1. All XRPD data reported herein were obtained using Cu Kα-1 radiation, having the wavelength 1.541 Å and were obtained using a Bruker AXS D8 Advance Powder X-ray Diffractometer.

Crystalline DC-cholesterol hydrochloride is characterized by an XRPD diffraction pattern comprising characteristic peaks at about 4.1, 6.1, 8.1, 14.1, 15.8, 17.3, 17.3, 17.5, 17.7, 18.3, 19.4, 24.1, and 24.4, ±0.2 degrees two theta.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

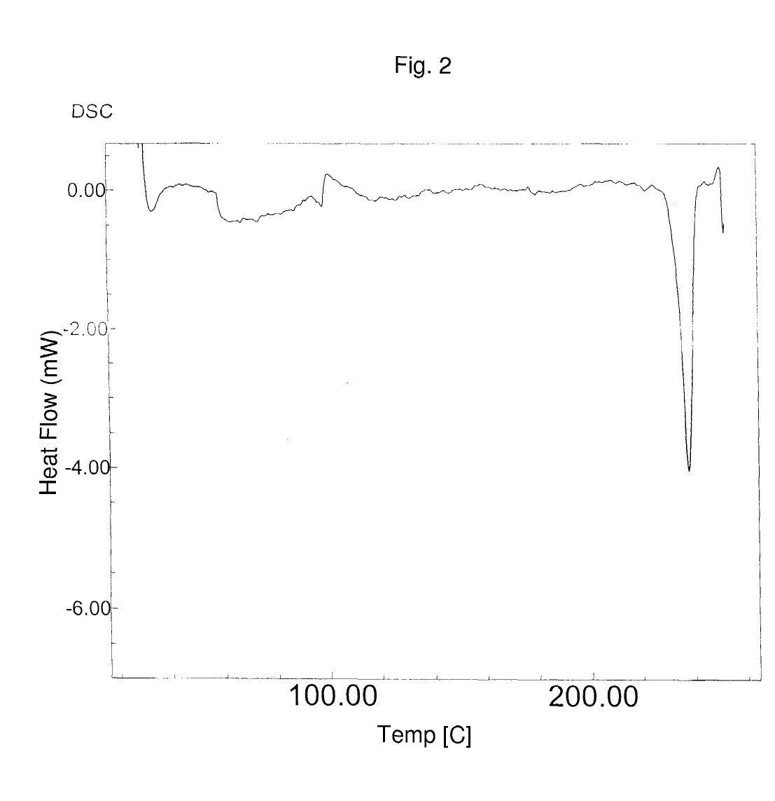

Crystalline DC-cholesterol hydrochloride of the present invention has a characteristic differential scanning calorimetric curve substantially in accordance with FIG. 2, having an endothermic peak at about 238° C. (Onset: 234° C. and Endset: 240° C.).

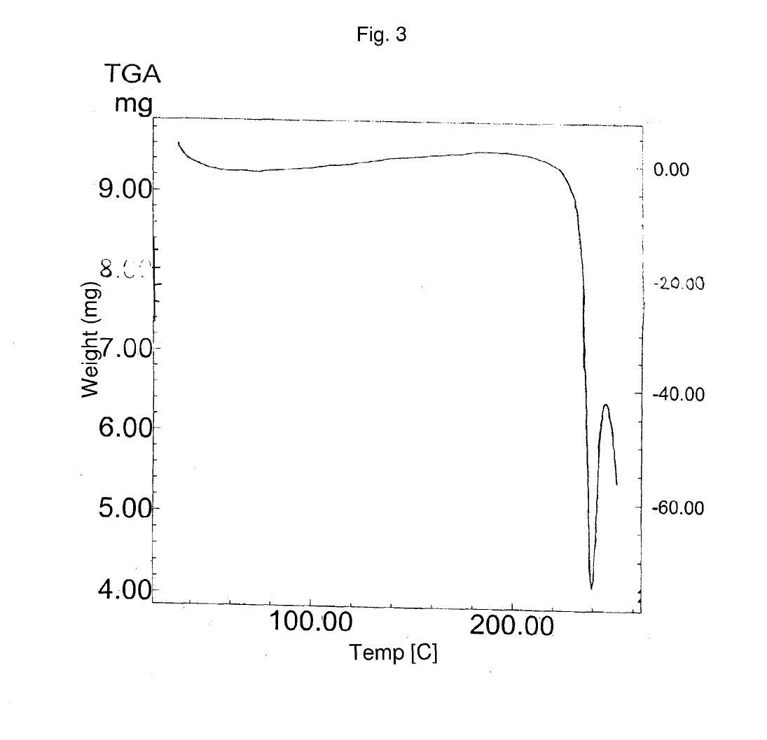

Crystalline DC-cholesterol hydrochloride of the present invention has a characteristic termogravimetric (TGA) curve corresponding to a weight loss of about 0.04% w/w substantially in accordance with FIG. 3.

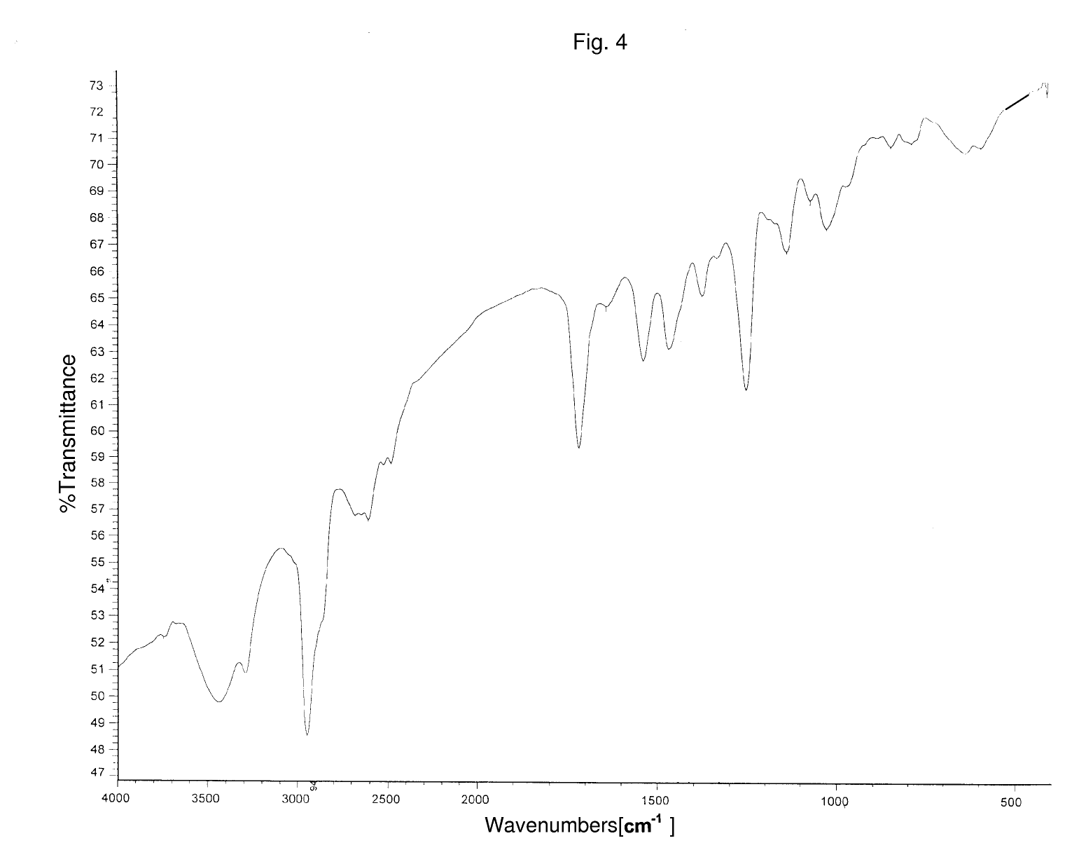

The infrared (IR) absorption spectrum of DC-cholesterol has been recorded on a Perkin Elmer System Spectrum 1 model spectrophotometer, between 450 cm⁻¹ and 4000 cm⁻¹, with a resolution of 4 cm⁻¹ in a potassium bromide pellet, the test compound being at the concentration of 1% by mass. Crystalline DC-cholesterol is characterized by an IR spectrum substantially in accordance with FIG. 4.

Certain specific aspects and embodiments of the invention will be explained in more detail with reference to the following examples, which are provided by way of illustration only and should not be construed as limiting the scope of the invention in any manner.

### EXAMPLE 1

### Preparation of DC-Cholesterol Using 1,1′-Carbonyldiimidazole

500 mg of cholesterol, 5 ml of dichloromethane and 600 mg of 1,1′-carbonyldiimidazole were charged into a clean and dry round bottom flask. The reaction mixture was stirred at 24° C. for 1 hour. The obtained solution was cooled to 2° C. and then a solution of N,N-dimethylethylenediamine (400 mg, 3.5 moles) in dichloromethane (2 ml) was added at 4° C. The reaction mixture was allowed to reach the temperature to 25° C. and then stirred at 25° C. for 1 hour. After completion of the reaction, the reaction mixture was washed with 10 ml of demineralized water and 10 ml of brine solution. The obtained solution was dried over 1.0 g of anhydrous sodium sulfate and the reaction solution was concentrated completely at 47° C. under a vacuum of 480 mm Hg to afford 400 mg of the title compound.

### EXAMPLE 2

### Preparation of DC-Cholesterol Using Triphosgene

500 mg of cholesterol, 10 ml of dichloromethane and 267 mg of triethylamine were charged into a clean and dry round bottom flask. 591 mg triphosgene in 5 ml of dichloromethane was added dropwise at 36° C. over about 20 minutes to the above reaction mixture. The reaction solution was stirred for about 2 hours at 25° C. and then 810 mg of N,N-dimethylethylenediamine was added to the reaction mixture over 10 minutes. The reaction mixture was stirred at 24° C. for 2 hours and the resultant reaction mixture was washed with 10 ml of demineralized water. The obtained solution was dried on 1.0 g of anhydrous sodium sulfate and then the solution was concentrated completely at 47° C. under a vacuum of 480 mm Hg to afford 450 mg of the title compound.

### EXAMPLE 3

### Preparation of DC Cholesterol

17.55 g of N,N-dimethylethylenediamine and 45 ml of dichloromethane were charged into a clean and dry round bottom flask. The resultant reaction solution was cooled to about 0° C. and then cholesteryl chloroformate solution (45 g of cholesteryl chloroformate in 225 ml of dichloromethane) was added to above reaction solution over about 30 minutes. The resultant reaction solution was allowed to reach a temperature of 30° C. and then was stirred for 30 minutes. The reaction solution was washed with 500 ml of demineralized water, then 200 ml brine solution, and was passed through anhydrous sodium sulphate. The resultant reaction solution was concentrated completely at about 47° C. under a vacuum of 480 mm Hg. 50 ml of n-hexane was charged to the residue and then distilled completely. Again 50 ml of n-hexane was charged to the residue and distilled until a 25 ml volume remained. The separated solid was filtered to afford 35.5 g of the title compound.

### EXAMPLE 4

### Preparation of DC-Cholesterol Hydrochloride

44.5 g of DC-cholesterol, prepared according to Example 3, and 445 ml of isopropyl alcohol were charged into a clean and dry round bottom flask and stirred for 10 minutes. 29.8 ml of hydrogen chloride in isopropanol (17.1% w/v) was added dropwise to the above reaction mixture over 20 minutes. The obtained reaction solution was immediately converted into a suspension and then the suspension was stirred for 1 hour at 26° C. The obtained suspension was filtered and the solid was washed with 89 ml of isopropyl alcohol. Finally, the solid was suction dried for 30 minutes and then dried for 6 hours under a vacuum of 600 mm Hg at 45° C. to afford 41.5 g of the title compound with a purity of 96.73% by HPLC.

FIGS. 1-4 show, respectively, the XRPD pattern, the DSC curve, the TGA curve, and the IR absorption spectrum for the product.

We claim:

1. A process for preparing 3β-[N-(N′,N′-dimethylaminoethane)-carbamoyl]cholesterol or a salt thereof, comprising reacting cholesterol, triphosgene, and N,N-dimethylethylenediamine in the presence of a base.

2. The process of claim 1, wherein reacting is conducted in a solvent comprising dichloromethane.

3. The process of claim 1, where in a molar ratio of cholesterol to N,N-dimethylethylenediamine is about 1:1 to about 1:8.

4. The process of claim 1, wherein a molar ratio of cholesterol to triphosgene is about 1:1 to about 1:3.

5. The process of claim 1, wherein a base comprises sodium hydroxide, potassium hydroxide, sodium carbonate, sodium bicarbonate, potassium carbonate, potassium bicarbonate, triethylamine, diethylisopropylamine, diisopropyl ethylamine, methylamine, or ammonia.

6. The process of claim 1, wherein 3β-[N-(N′,N′-dimethylaminoethane)-carbamoyl]cholesterol or a salt thereof has a purity at least about 95% by weight.

7. A process for preparing 3β-[N-(N′,N′-dimethylaminoethane)-carbamoyl]cholesterol or a salt thereof comprising reacting cholesterol, 1,1′-carbonyldiimidazole, and N,N-dimethylethylenediamine.

8. The process of claim 7, wherein reacting is conducted in a solvent comprising dichloromethane.

9. The process of claim 7, wherein a molar ratio of cholesterol to N,N-dimethylethylenediamine is about 1:1 to about 1:5.

10. The process of claim 7, wherein a molar ratio of cholesterol to 1,1′-carbonyldiimidazole is about 1:1 to about 1:5.

11. The process of claim 7, wherein 3β-[N-(N′,N′-dimethylaminoethane)-carbamoyl]cholesterol or a salt thereof has a purity at least about 95% by weight.

12. A process for preparing 3β-[N-(N′,N′-dimethylaminoethane)-carbamoyl]cholesterol or a salt thereof comprising reacting cholesteryl chloroformate with N,N-dimethylethylenediamine, wherein a molar ratio of cholesteryl chloroformate to N,N-dimethylethylenediamine is about 1:1 to about 1:5.

13. The process of claim 12, wherein reacting is conducted in a solvent comprising dichloromethane, dichloroethane, or chloroform.

14. The process of claim 12, wherein reacting is conducted in a solvent comprising dichloromethane.

15. The process of claim 12, further comprising reacting 3β-[N-(N′,N′-dimethylaminoethane)-carbamoyl]cholesterol with hydrochloric acid in a solvent comprising an ether, a hydrocarbon, an alcohol, a ketone, or a nitrile.

16. The process of claim 12, wherein 3β-[N-(N′,N′-dimethylaminoethane)-carbamoyl]cholesterol or a salt thereof has a purity at least about 95% by weight.
